(19) United States
(12) Patent Application Publication
(10) Pub. No.: US 2010/0324507 A1
(43) Pub. Date: Dec. 23, 2010

(54) MICRO-AND/OR NANO-STRUCTURED PROTECTIVE OR PROCESS FILM

(75) Inventor: Stephan Maier, Leverkusen (DE)

Correspondence Address: FROMMER LAWRENCE & HAUG, 745 FIFTH AVENUE- 10TH FL., NEW YORK, NY, 10151, US

(73) Assignee: LTS Lohmann Therapie-Systeme AG, Andermach (DE)

(21) Appl. No.: 12/865,488

(22) PCT Filed: Jan. 8, 2009
(86) PCT No.: PCT/EP09/00048

§ 371 Date: Jul. 30, 2010

(30) Foreign Application Priority Data
Jan. 30, 2008 (DE) 10 2008 006 787.3

(51) Int. Cl. A61M 35/00; B32B 3/30

(52) U.S. Cl. 604/289; 428/156

(57) ABSTRACT

The invention relates to a protective or process film, produced of a thermoplastic material for use in the production of laminates of medical technology and to a laminate which comprises an adhesive-containing transdermal therapeutic system and a protective or process film adhering to the adhesive of the transdermal therapeutic system with a surface. The protective or process film comprises at least one surface that has a plurality of recesses and/or a plurality of non-recessed portions. The distance of two adjacent recesses and/or the distance of two adjacent non-recessed portions is less than five times the film thickness. The depth of the recesses is not less than 1.2 nanometer and not more than 95% of the film thickness. The film according to the invention effectively prevents the adhesive from sticking to the protective or process film.

The invention relates to a protective or process film made of a thermoplastic material for use in the production of medical laminates, and also to a laminate comprising an adhesive-containing transdermal therapeutic system, and a protective or process film that attaches to the adhesive of the transdermal therapeutic system by one surface.

In the production of medical laminates, a group which includes, for example, ready-to-use transdermal therapeutic systems, there are different kinds of films in use. One kind of film, used, for example, as a backing film, adheres permanently to an adhesive-containing layer of a transdermal therapeutic system. Another kind of film, used, for example, as a protective and process film, is to be removable without residue from the adhesive-containing layer following its use. The last-mentioned films are dehesive films.

To date, protective and process films in transdermal therapeutic systems have been provided with dehesive coatings, examples being silicone or fluoropolymer coatings. In this case a base film is coated with a second material, which must be taken into account when the transdermal therapeutic system is formulated. A coating of this kind requires pretreatment of the film surface, by means of a corona pretreatment, for example, which generates polar groups on the surface of the base film, the material of the coating attaching firmly to these groups. One of the risks of this costly and inconvenient process is that of insufficient adhesion of the coating to the base film, as a result of inadequate pretreatment. In the course of the coating operation, moreover, there may be fluctuations in the application rate of the coating, possibly going as far as to produce defect sites. This may result locally in high adhesion forces, thereby disrupting the controlled progress of the process of laminate production and/or robbing the transdermal therapeutic system of its functionality.

The problem on which the present invention is based is therefore that of effectively preventing the sticking of an adhesive to the protective and process film.

This problem is solved with the features of the main claim. For that purpose, the protective or process film comprises at least one surface which has a multiplicity of recesses and/or a multiplicity of non-recessed regions. The distance between adjacent recesses and/or the distance between two adjacent non-recessed regions is less than five times the film thickness. Additionally, the depth of the recesses is not less than 1.2 nanometers and not more than 95% of the film thickness.

Further details of the invention will become apparent from the dependent claims and from the description, given below, of embodiments which are represented schematically.

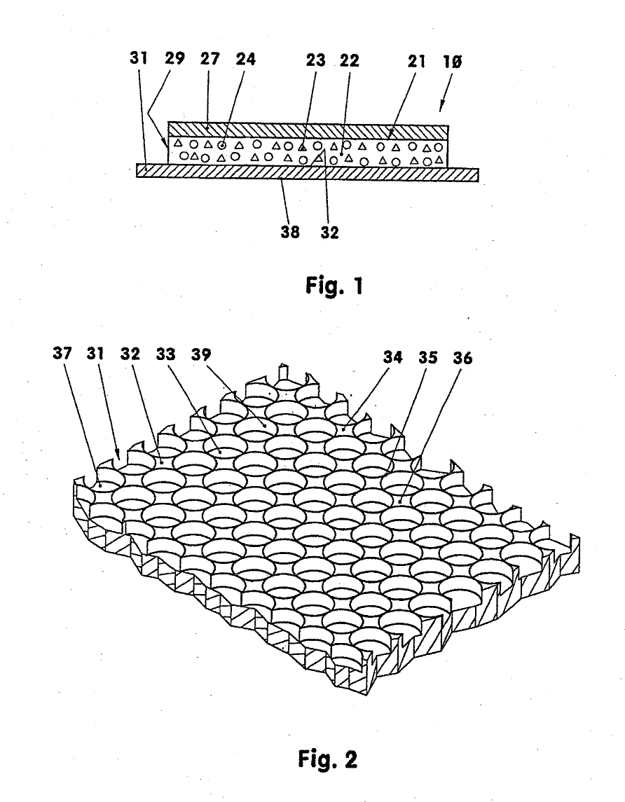

FIG. 1: laminate comprising a transdermal therapeutic system and a protective film;

FIG. 2: detail of a protective or process film;

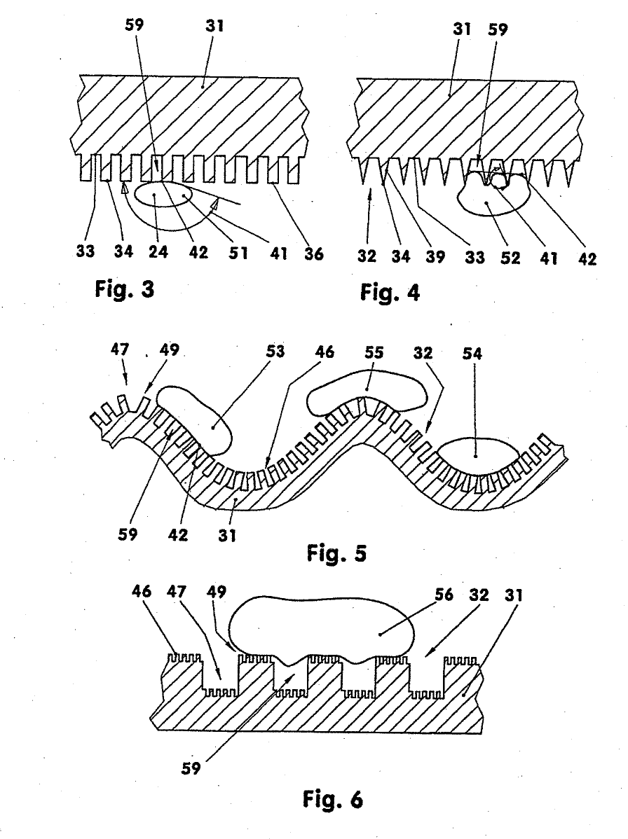

FIG. 3: detail of a drop of adhesive on a protective or process film;

FIG. 4: pointed structures of the protective or process film;

FIG. 5: micro- and nano-structured protective or process film;

FIG. 6: variant of the micro- and nano-structured protective or process film;

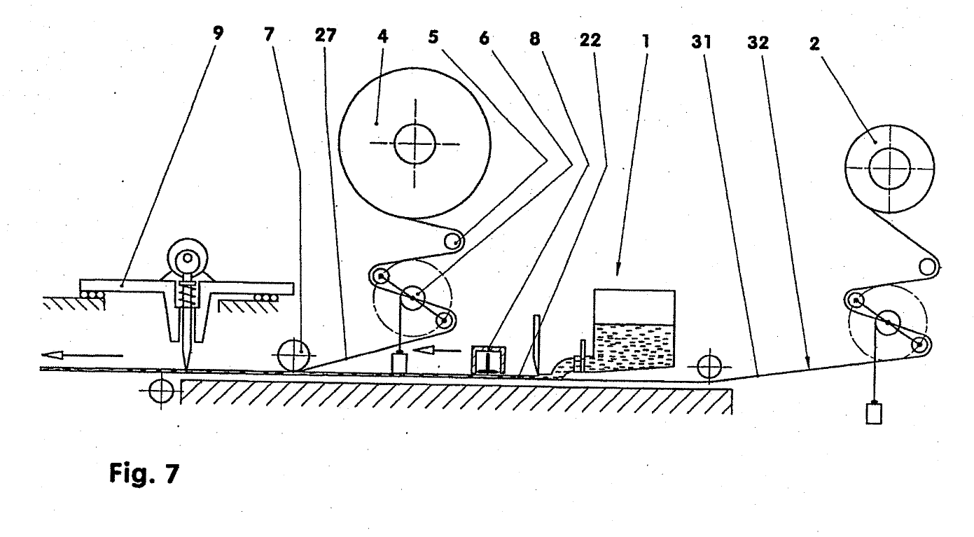

FIG. 7: production of a laminate with protective film;

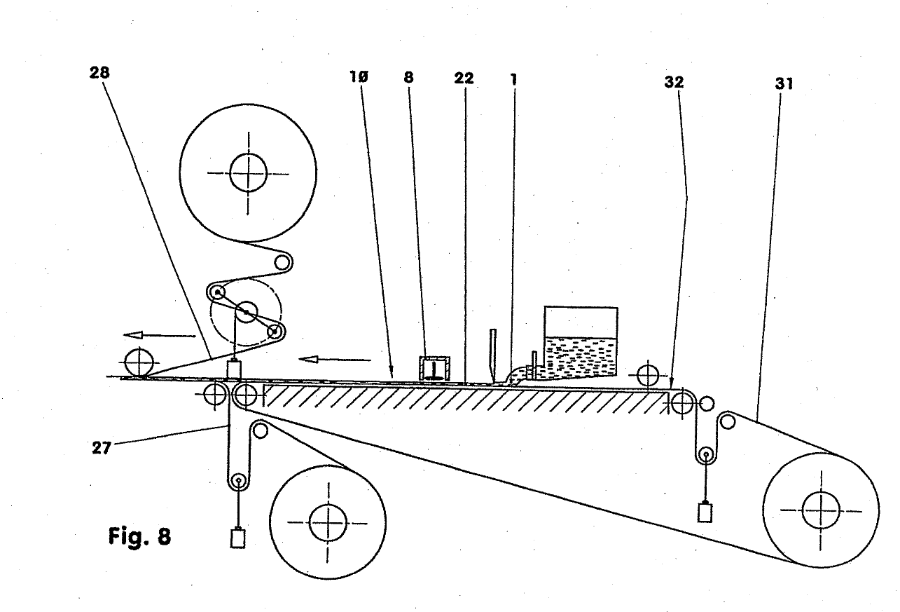

FIG. 8: production of a laminate on a process film.

FIG. 1 shows a laminate (10) which comprises a transdermal therapeutic system (21) and a protective film (31). A laminate (10) of this kind is, for example, a ready-to-use patch removed from the packaging. Immediately prior to application, the patient must remove the protective film (31) in order to allow the transdermal therapeutic system (21) to be attached to the skin by an adhesive-containing layer (22).

The transdermal therapeutic system (21) is, for example, an active ingredient patch (21) featuring an adhesive matrix. The active ingredient (23) and the adhesive (24) are arranged, for example, in a joint adhesive-containing layer (22) on a backing film (27). Alternatively, the adhesive (24) may be arranged in a layer, separate from the active ingredient (23), of the transdermal therapeutic system (21), which is of multi-layer construction, for example. In a plan view of the transdermal therapeutic system (21), the backing film (27) and the active ingredient and adhesive layer (22) have, for example, the same size. Located beneath the active ingredient and adhesive layer (22) in the exemplary embodiment of FIG. 1 is the protective film (31), which attaches, for example, to the active ingredient and adhesive layer (22). Attachment means that the protective film (31) can be parted with little force, by hand, and without residue, from the active ingredient and adhesive layer (22). The specific application of force required for peeling off the film is, for example, less than 5 newtons per 25 millimeters of film width. In contrast to attachment, adhesive bonding produces a firm bond which can be parted rarely without residue and only with substantial application of force, as for example with a specific force greater than the aforesaid force value.

At the edges (29), for example, the protective film (31) juts out beyond the active ingredient and adhesive layer (22). In the case of an embodiment of the laminae (10) having a plurality of layers or a separate active ingredient (23) and adhesive (24), the protective film (31) is arranged on the adhesive-containing layer (22) facing away from the backing film (27). The laminate (10) may also be implemented without active ingredient (23).

The adhesive (24) which ensures adhesion to the skin of the patient when the transdermal therapeutic system (21) is applied is—for example—pressure-sensitive. The adhesive (24) is composed substantially, for example, of a matrix-forming pressure-sensitive adhesive. For this purpose it is possible to use, for example, polyacrylates, silicones, polyisobutylenes, rubber, rubberlike synthetic homopolymers, copolymers or block polymers, butyl rubber, styrene/isoprene copolymers, polyurethanes, copolymers of ethylene, polysiloxanes, or styrene/butadiene copolymers, individually and/or in combination. The adhesive (24) may also, however, comprise further substances, such as, for example, physiologically active substances, dyes, plasticizers, tackifiers, permeation enhancers, etc. The surface tension of the adhesive (24) relative to its vapor phase amounts, for example, to between 30 and 50 millinewtons per meter.

The protective film (31) in the exemplary embodiment is composed of a thermoplastic material, e.g., of polyester, polyethylene, polypropylene, etc. This film (31) is, for example, of aroma-proof, water-proof and/or oxygen-proof design. Its thickness amounts, for example, to a tenth of a millimeter. At least the—for example—non-polar surface (32) of the protective film (31) has no silicone or fluoropolymer coating.

In the case of a smooth surface, the stated thermoplastic material has, for example, a surface tension which is equal to the surface tension of the adhesive (24) used or deviates from that value by not more than 20%, for example. The adhesive (24) and a smooth surface of the film material therefore have a strong tendency to stick to one another.

In the case of the protective film (31) in FIG. 1, its surface (32) facing the transdermal therapeutic system (21) has recesses (33) and non-recessed regions (34). One example of a structure of this kind is shown by FIG. 2. The structure shown there as an extract has cylindrical recesses (33) which are surrounded by a latticelike non-recessed region (34). The depth of the recesses (33) amounts, for example, to 100 nanometers. It can be between 1.2 nanometers and 95% of the film thickness. The diameter of the recesses (33) shown here amounts, for example, to 50 nanometers. It may, for example, be up to five times the film thickness.

The lattice rods (35) of the non-recessed regions (34) in this case have a thickness, for example, of not more than 50 nanometers, and so, in this exemplary embodiment, two adjacent recesses (33) have a distance of 50 nanometers from one another. This distance as well may be up to five times the film thickness. The end face (36) is, for example, a planar face.

In the exemplary embodiment, the multiplicity of recesses (33) and of the non-recess regions (34) are arranged regularly. The structure may, however, also have an irregular arrangement; the depths of the recesses (33) may be different. The base areas (37) may be planar, concave, convex, etc., in form.

In the case of a structure having a multiplicity of non-recessed regions (34), these regions may take the form, for example, of rods having a square, round, rectangular, triangular, etc., cross section. In that case the end faces (36) may be of planar or convex form. The non-recessed regions (34) may be conical or pyramidal, mushroom-shaped, etc., in form.

The structuring of the surface (32) produces a resultant effective surface which is in contact with the adhesive-containing layer (22) and has properties which differ from the properties of the base material. For example, with respect to a drop of adhesive which is applied, a surface (32) with such structuring has a significantly lower surface energy than a smooth, uncoated base material. As a result of this it is possible, for example, for adhesive bonds to form only to a small degree between the adhesive (24) and the protective film (31). Sticking of the protective film (31) to the adhesive-containing layer (22) and/or to the transdermal therapeutic system (21) is therefore effectively prevented.

The surfaces (38) of the protective film (31) that are situated on the side facing away from the transdermal therapeutic system (21) may be smooth or structured.

The laminate (10) with a transdermal therapeutic system (21) is produced, for example, in a multi-stage, inter-linked operation; cf. FIG. 7. For example, at an application station (1), the adhesive-containing composition, which may also contain active ingredient, is first applied as a layer (22) directly to the protective film (31), which is unwound from a roll (2). The protective film (31) here serves as a transport film and is conveyed through the production apparatus. After drying, the reduction of the moisture content of the solvent-containing composition, which is accomplished, for example, by means of a drying station (9), or, in the case of hotmelt adhesives, after the cooling of the adhesive composition, the exposed surface of the adhesive matrix is covered over its full area with further layers and/or with the backing film (27). This produces an active ingredient-containing or active ingredient-free, single-layer or multi-layer, intermediate laminate product.

Alternatively, the adhesive-containing composition may also be coated onto the backing film (27). In this case it is the backing film (27) which is the transport film used to convey the intermediate product through the production apparatus. After the adhesive-containing layer (22) has dried or cooled, it is then covered over its full area with further layers and/or with the protective film (31). In this case the protective film (31) is placed onto the last adhesive-containing layer (22) in such a way that the structured surface (32) faces the adhesive-containing layer (22).

Using a single-stage or multi-stage punching apparatus (9), the resulting intermediate laminate product is punched to produce the individual laminate (10), in the form, for example, of a laminate section (10). Appropriate contour punching of the patch (21) and removal of the projecting edge have the effect, for example, that the protective film (31) juts out all round beyond the active ingredient and adhesive layer (22). The separated laminate section (10) prepared in this way is then conveyed to a packing station and packaged, for example, into a packaging unit.

In the as-supplied state, the protective film (31), with a length, for example, of several hundred meters and a width, for example, of between, say, 10 mm and 7000 mm, is wound up, for example, on a roll (4). From this roll (4)—this roll (4) is in this case, for example, between 500 mm and 2500 mm wide—the protective film (31) is guided, for example, via a support roller (5) and a weighted pendulum roller (6), to a pressing roller (7).

Before the adhesive composition is applied to the protective or process film (31), or before the adhesive layer is covered by the protective or process film (31), the surface (32) facing the adhesive layer (22) is prepared. This may even take place outside the adhesive coating apparatus. Over the entire area, for example, a structure is applied to the surface (32) of the protective film (31) by means of injection molding, thermoplastic impression, or rolling. Application of the structure by means of substrate deposition is also conceivable.

The base structure to be applied may be generated, for example, by means of holographic recording methods. Such methods are implemented, for example, using the technology of two-beam interference on the basis of coherent optical systems, or electron-beam systems. In this operation, for example, a glass plate coated with photoresist is introduced into an interference pattern produced by laser beams. The exposure produces a pattern on the resist, in which the distances are situated, for example, in the nanometer range. With the aid of glass plates made conductive by metalizing, it is possible, by electroforming or by galvanic replication, for example, using nickel deposition, to produce copies of the structured surface. These copies can be produced in the form of plates or thin nickel sheets. The forms can then be transferred to the protective film by means of injection molding, thermoplastic impression, or by means of rolling.

In the production of a laminate section (10), composed, for example, of a backing film (27), an active ingredient and adhesive layer (22), and a protective film (31), one of the factors determining the resultant release force for the removal of the protective film (31) is that of whether the coating of the adhesive composition has taken place directly onto the structured protective film (31), or whether the structured protective film ((31) has been laminated onto the adhesive composition only after the drying or cooling of said composition. Where the adhesive composition is coated directly onto the protective film, the liquid adhesive composition, depending on its viscosity, is able to penetrate, for example, more deeply into the structure of the surface (32) than is the case with the laminating of the protective film (31) onto the dried or cooled, and highly viscous, adhesive layer. The forces needed to remove the protective film (31) from the adhesive (24) may be higher in the first case than in the second case.

As soon as the protective film (31) contacts the adhesive (24), the adhesive (24) conforms to the non-recessed regions (34) of the protective film (31); cf. the sectional representation in FIG. 3. In view of the small area of contact—in this exemplary embodiment, corresponding in each case to a section of the end face (36) of a non-recessed region (34)—no adhesive (24) is left hanging on the protective film (31) when the protective film (31) is removed from the active ingredient and adhesive layer (22). The transdermal therapeutic system (21) can be removed without residue from the protective film (31).

Shown in FIG. 3, for example, is a drop (51) of adhesive. The behavior of this drop is exactly the same as that of a layer of adhesive which is applied, for example, two-dimensionally to the structured surface (32) of the protective film (31). Following its application to the surface (32) of the protective film (31), the drop (51) of adhesive undergoes contraction. For example, a contact angle (41) of 160 degrees is formed between the drop (51) of adhesive and the film (31). The drop (51) of adhesive conforms only loosely to the protective film (31) or attaches to it lightly.

Owing to the described micro-structuring and/or nano-structuring of the surface (32), the physical properties of the bond of the protective film (31) to the adhesive (24), for example, are influenced. Thus, as compared with full-area application of adhesive, there is a substantial reduction in the resultant effective surface, and hence the effective surface energy of the film surface (32) is also reduced as compared with the unstructured material. As a result of this reduction in surface energy, only a weak adhesive bond is developed between the protective film (31) and the adhesive (24). Sticking of the two materials is prevented.

FIG. 4 shows a drop (52) of adhesive which extends over a plurality of non-recessed regions (34)—which in this case, for example, are of pyramidal form. In regions, the drop (52) of adhesive has penetrated into the recesses (33). Above the drop of adhesive (52), in the recesses (33), an air cushion (59) has formed that prevents further penetration of the drop (52) of adhesive into the recess (33). The depth of the recess (33) above the drop (52) of adhesive is greater than the range of the chemical and of the physical adhesive forces between the materials. The range of the last-mentioned forces is situated, for example, at between 0.2 nanometer and one nanometer. The depth of the recesses (33) amounts to at least 1.2 nanometers.

On this effective surface (42) composed of air cushions (59) and of non-recessed regions (34)—surfaces of this kind are referred to, for example, as composite surfaces—the attachment of the drop (52) of adhesive is low. Within the recesses (33), for example, it forms a contact angle of 160 degrees, for example, with the flanks (39) of the non-recessed regions (34). This property of the surface structure is needed especially when the protective film (31) is coated directly with the liquid adhesive composition.

FIG. 5 shows a protective or process film (31) having a microstructure (47) and having a nanostructure (46), with a number of drops (53-55) of adhesive located thereon. In the section shown, for example, the microstructure (47) has the form of a sinusoidal curve. In this exemplary embodiment, the distance between the individual maxima (49)—which is, for example, between one micrometer and five times the film thickness—is sufficiently large to allow the drops (53-55) of adhesive to follow the contour. Along the microstructure (47), a nanostructure (46) has been made in the film (31). The distance between the individual recesses (33) of the nanostructure (46) is less, for example, than one micrometer. Additionally, the distance between the non-recessed regions (34) of the nanostructure (46), shown in the sectional representation of FIG. 5, is less, for example, than one micrometer. The construction of the nanostructure (46) is, for example, like that described in connection with FIGS. 1 to 3. The protective or process film (31) may also be provided only with a microstructure (47) or only with a nanostructure (46).

The air cushions (59) in the recesses (33) prevent excessive attachment and bonding of the drop (53) of the adhesive to the protective or process film (31). FIG. 6 shows a protective or process film (31) having a microstructure (47) and a nanostructure (46), in which the wavelength of the microstructure (47) is of a shorter form than in the representation of FIG. 5. The drop (56) of adhesive does not follow the contour of the microstructure (47), but instead lies only on its maxima (49). A protective film (31) having an overlaid structure of this kind has particularly good dehesive properties. If in spite of this, as a result, for example, of temperature or pressure influences, a drop (56) of adhesive were to follow the contour of the microstructure (47), the overlaid nanostructure (46) prevents sticking of the drop (56). The overlaying of microstructures and nanostructures makes it possible to produce, for example, protective or process films (31) having properties of graduated dehesiveness. The release force needed to remove the protective or process film (31) from the adhesive layer (22) can be tailored to the particular application by means of an appropriately selected structuring.

FIG. 8 shows a dehesive film (31) having the properties and the geometric surface construction of the above-described protective film (31) in use as a process film (31). This process film (31) is, for example, a continuously conveying, circulating film (31), on which the adhesive composition is applied as a viscous solution at an applicator mechanism (1). In a downstream station (8) for drying the laminate (10), the moisture content is reduced to a setpoint value. The adhesive-containing layer (22) thus prepared is then, pushed down, for example, by the process film (31) and pushed, for example, onto a backing film (27). A further outer film (28) is supplied, for example, on the side facing away from the backing film (27). This outer film (28) may, on its side facing the adhesive-containing layer (22), have the same surface structure as the process film (31).

In the course of the drying and withdrawal of the adhesive-containing layer (22), there are no residues of the adhesive (24) left on the process film (31), and so, on the next circulation of the film (31), the new layer—for example, active ingredient and adhesive layer (22)—is not adversely affected.

The geometric design of the surface (32) of the process film (31) may correspond to the design described in connection with FIGS. 2-6. A process film (31) of this kind can also be used, for example, for the short-term storage of a laminate (10) or of part of a laminate (10). The surface (32) of the dehesive process film (31) that faces the adhesive-containing layer (22) is produced, for example, as described in connection, with the first exemplary embodiment.

Combinations of the exemplary embodiments described are also conceivable.

### LIST OF REFERENCE NUMERALS

1 active ingredient and adhesive application, application station
2 roll
4 roll
5 support roller
6 pendulum roller
7 pressing roller
8 drying station
9 punching apparatus
10 laminate, laminate section
21 transdermal therapeutic system, patch
22 adhesive-containing layer, active ingredient and adhesive layer
23 active ingredient
24 adhesive
27 backing film
28 outer film
29 edges
31 dehesive film, protective or process film
32 internal surface of (31)
33 recesses
34 non-recessed regions
35 lattice rods
36 end faces
37 base areas
38 outer surface
39 flanks
41 contact angle
42 effective surface
46 nanostructure
47 microstructure
49 maxima of (47)
51-56 drops of adhesive
59 air cushion

1. A protective or process film (31) made of a thermoplastic material for use in the production of medical laminates (10), characterized
in that it has at least one surface (32) which has a multiplicity of recesses (33) and/or a multiplicity of non-recessed regions (34),
in that the distance between two adjacent recesses (33) and/or the distance between two adjacent non-recessed regions (34) is less than five times the film thickness, and
in that the depth of the recesses (33) is not less than 1.2 nanometers and not more than 95% of the film thickness.

2. The protective or process film (31) of claim 1, characterized in that the structuring marked by the recesses (33) is made over the full area in this surface (32).

3. A medical laminate (10) comprising an adhesive-containing transdermal therapeutic system (21), and a protective or process film (31) attaching to the adhesive (24) of the transdermal therapeutic system (21) by one surface (32), characterized in that
the protective or process film (31) is made of a thermoplastic material,
in that the stated surface (32) has a multiplicity of recesses (33) and/or a multiplicity of non-recessed regions (34),
in that the distance between two adjacent recesses (33) and/or the distance between two adjacent non-recessed regions (34) is less than five times the film thickness, and
in that the depth of the recesses (33) is not less than 1.2 nanometers and not more than 95% of the film thickness.

4. The laminate (10) of claim 3, characterized in that the adhesive (24) is pressure-sensitive.
